(19) United States
(12) Patent Application Publication
(10) Pub. No.: US 2026/0245720 A1
(43) Pub. Date: Aug. 20, 2026

(54) CAREGIVER AVATAR SYSTEM

(71) Applicant: ELYSIAN SOFTECH LTD., Petah Tikva (IL)

(72) Inventors: Adrian Paz, Petah Tikva (IL); Tomer Paz, Petah Tikva (IL)

(73) Assignee: ELYSIAN SOFTECH LTD., Petah Tikva (IL)

(21) Appl. No.: 19/161,249

(22) PCT Filed: Mar. 19, 2024
(86) PCT No.: PCT/IL2024/050284

§ 371 Date: Aug. 31, 2025

Related U.S. Application Data
(60) Provisional application No. 63/453,641, filed on Mar. 21, 2023.
(60) Provisional application No. 63/527,004, filed on Jul. 15, 2023.
(60) Provisional application No. 63/537,487, filed on Sep. 9, 2023.

(51) Int. Cl. G16H 40/67; G06F 40/40; G16H 10/20; G16H 80/00

(52) U.S. Cl. CPC G16H 40/67; G16H 10/20; G16H 80/00; G06F 40/40

(57) ABSTRACT

A caregiver avatar system for caregiver-patient interactions comprising an AI system with a virtual avatar, wherein the avatar is configured to handle automatic or semi-automatic interactions between a caregiver and a patient. The system and method are configured to enhance healthcare delivery and/or improve medical practices without replacing the caregiver.

### RELATED APPLICATIONS

This application is a continuation-in-part of PCT Patent Application No. PCT/IL2024/050284, filed on 19 Mar. 2024, which claims the benefit of priority under 35 USC § 119(e) of U.S. Provisional Patent Application No. 63/527,004 filed on 15 Jul. 2023, U.S. Provisional Patent Application No. 63/537,487 filed on 9 Sep. 2023, U.S. Provisional Patent Application No. 63/453,641 filed on 21 Mar. 2023, the contents of which are incorporated herein by reference in their entirety.

### FIELD AND BACKGROUND OF THE INVENTION

The present invention, in some embodiments thereof, relates to a caregiver avatar system and, more particularly, but not exclusively, to enhance healthcare delivery and/or medical communication.

With the continued growth of the world population, the current shortage of medical care is expected to increase. While caregivers intend to provide the best medical care the can, their time and resources are limited. Additionally, naturally, caregivers want to maximize their revenue potential.

Therefore, there is a critical gap in the healthcare industry, which necessitates the adoption of advanced technologies to enhance healthcare delivery.

### SUMMARY OF THE INVENTION

According to an aspect of some embodiments of the invention, there is provided a system for interactions between a caregiver and a subject including: a remote interface for the subject; a remote interface for a caregiver; a processor to control an avatar including a Large Language Model (LLM) for conversation and an Artificial Intelligence (AI) module for instructing the LLM to instruct the LLM and to control a virtual avatar to simulate the caregiver interactions with a subject including collecting clinical data and evaluating collected data for medical conditions.

According to some embodiments of the invention, caregiver includes at least one of a physician, medical assistant, psychologist, behavior therapy specialist, social worker, wellbeing specialist, general health specialist, preventive medicine specialist, antiaging specialist, cosmeticians.

According to some embodiments of the invention, the avatar is configured to realistically resemble any particular caregiver of a group of caregivers.

According to some embodiments of the invention, the processor is configured to simulate questions and responses of each particular caregiver.

According to some embodiments of the invention, the questions and responses concern at least one of symptoms, diseases, medications, treatments, tests.

According to some embodiments of the invention, the avatar is customizable to include a voice of the caregiver, intonations, sentence structure, word choices, expressions, facial expressions, mannerisms, gestures, lip movements, or a combination thereof.

According to some embodiments of the invention, the avatar is configured to appear in a video call with appropriate facial expression and voice intonations.

According to some embodiments of the invention, the avatar is configured for text communication with appropriate sentence structure, word choices, and expressions to simulate the caregiver.

According to some embodiments of the invention, the processor is configured to simulate the avatar conversing in a language different from an interface language used by at least one of the subjects and the caregiver.

According to some embodiments of the invention, the processor is configured to present and record the conversation as subtitles in real time.

According to some embodiments of the invention, the system includes a caregiver interface including tools to monitor, assess, and interrupt subject-avatar interactions in real-time.

According to some embodiments of the invention, the AI is configured to present caregiver interactions with the subject that appear the same as avatar interactions with the subject.

According to some embodiments of the invention, the AI system is configured to present caregiver interactions with the subject to appear different to avatar interactions with the subject.

According to some embodiments of the invention, the AI system is configured to receive simple instructions from the caregiver and in response to the simple instructions trigger complex avatar actions chosen from a group including writing prescriptions and sending them to the subject, scheduling tests, scheduling procedures, scheduling a visit with the avatar, scheduling a visit with the caregiver, sending a medication to the subject, sending an article of medical equipment to the subject, sending a secondary caretaker to the subject, sending another caregivers to the subject, scheduling physiotherapy, scheduling a medical test, managing a medical test, managing blood sampling and blood testing, organizing transportation to or from a medical facility.

According to some embodiments of the invention, the clinical data includes at least one of, a medical history, an electronic medical record, a hard copy medical record, current health issues, symptoms, test results, remote sensor readings, appearance of skin, body shape, appearance of eyes, appearance of a mouth, facial features, a facial expression, a mood, a behavior, a speech parameter, a breathing parameter, a sound and a gait.

According to some embodiments of the invention, the processor is configured to supervise the subject performing at least one home task in front of at least one of a camera and a microphone, the at least one home task chosen from: walking, climbing stairs, moving a body part, performing a facial gesture, saying a specific sentence.

According to some embodiments of the invention, the processor is configured to supervise the subject performing at least one home task to measure a medical parameter selected from: hrv, blood pressure, eye pressure, temperature, o2 saturation, blood glucose, breathing gases, respiratory volumes, breathing sounds, heart sounds, skin sympathetic activity, ecg, eeg.

According to some embodiments of the invention, the system further includes at least one of a remote sensor, a home tasking device, a cell phone sensor used for performing the at least one home tasks.

According to some embodiments of the invention, the system further includes at least one of wherein the processor is configured to guide and monitor the subject performing at least one task using at least one of a remote camera, a microphone, a gps, a proximity sensor, a hrv sensor, an accelerometer, a stepping sensor, a gyroscope, a blood gases sensor, a glucose sensor, a sympathetic activity sensor, a sweat sensors, a breathing volume sensor, a gas sensor, a metabolic rate sensor, a blood pressure tester, an ecg, an eeg, an electromyography sensor, a bioimpedance sensor.

According to some embodiments of the invention, the home task includes at least one task chosen from a group consisting of physical activity, walking, dressing, undressing, urination, defecation, appetite evaluation, eating behavior evaluation, breathing exercise, meditation, physiotherapy exercise, dexterity exercise, memory test, a behavior therapy task, a cognitive therapy task, filling a log, filling a diary, taking medications under direct supervision, developing an interpersonal skills, evaluating an emotion during different scenarios, eye tracking.

According to some embodiments of the invention, the system further includes a questionnaire provided to the subject to evaluate at least one of treatment satisfaction, functional status, anxiety level, depression level, level understanding of the medical conditions, satisfaction with medical education received, satisfaction with availability of care, satisfaction with provision of specialists consultations, satisfaction with provision of ancillary medical services such as transportation, scheduling of visits tests and procedures, home delivery of medication, medical equipment and availability of home visits of different caretakers, cost effectiveness and level of redundancy of services.

According to some embodiments of the invention, the AI system is further configured to receive limits on actions performed without approval of the caregiver.

According to some embodiments of the invention, the remote interface for the caregiver is configured to facilitate direct participation of the caregiver in a conversation between the subject and the avatar.

According to some embodiments of the invention, the remote interface for the caregiver is configured to facilitate silencing the avatar while the caregiver converses with the subject.

According to some embodiments of the invention, the remote interface for the caregiver is configured to facilitate a caregiver to participate and monitor conversations with a plurality of subjects simultaneously.

According to some embodiments of the invention, the remote interface for the caregiver is configured to facilitate a plurality of caregivers to converse with the avatar during the same conversation regarding a particular subject.

According to some embodiments of the invention, the remote interface for the caregiver is configured to facilitate the caregiver conversing with the avatar to add more information regarding the subject from a direct interaction between the subject and the caregiver.

According to some embodiments of the invention, the avatar provides information to the caregiver regarding medical guidelines, treatment options and algorithms, diseases and scientific data.

According to some embodiments of the invention, the avatar provides to the patient existing movies, or generates new short instruction movies or games, regarding diseases, treatments, physiotherapy exercises, home tasks, and procedures according to the data present in guidelines or to textual or verbal instructions from the caregiver.

According to some embodiments of the invention, the remote interface for the caregiver is configured to facilitate recording a face to face or virtual conversation of the subject with the caregiver and identify who the what and add this data to a summary accordingly.

According to some embodiments of the invention, the remote interface for the caregiver is configured to present a summary of information regarding the subject, avatar and avatar physician conversation in predetermined formatted form according to predefined headings.

According to some embodiments of the invention, recorded data is secured from being modified and is kept and accessed in a secure way.

According to some embodiments of the invention, the remote interface for the caregiver is configured to facilitate data retrieval regarding one or more subjects in formatted way according to predefined headings.

According to some embodiments of the invention, the AI system is prevented from prescribing treatment to the subject without approval of the caregiver.

According to some embodiments of the invention, the AI system is prevented from suggesting tests or actions that may be dangerous to a particular subject.

According to an aspect of some embodiments of the invention, there is provided a method for a caregiver-subject interface, the method including: collecting pre-visit subject data; summarizing patent data for a caregiver, wherein the summary includes a proposed diagnosis and recommendations for future actions; assisting in the subject visit; and ensuring follow-up care of the subject.

According to some embodiments of the invention, the collecting subject data includes actively interviewing the subject, administering tests to the subject, collecting data from subject associated devices, and accessing medical records of the subject.

According to some embodiments of the invention, the assisting includes monitoring caregiver instructions and warning when the caregiver appears to be acting in a manner which is not in accordance with accepted practice or contravenes medical guidelines.

According to some embodiments of the invention, the assisting includes recording visits for later analysis.

According to some embodiments of the invention, the follow-up care includes at least one of monitoring a condition of the subject, monitoring subject compliance, scheduling follow up visits, scheduling consultations, scheduling tests, ordering transportation, renewing prescriptions, answering patent inquiries regarding their diagnosis, prognosis, and treatment.

According to some embodiments of the invention, the method further includes alerting the caregiver to a possibly dangerous health situation.

According to an aspect of some embodiments of the invention, there is provided a method for dynamic injection instruction to an AI avatar system, the method including: accessing a specific library of instructions from a plurality of libraries in accordance to a specific prompt file of predetermined conversation cues; and giving a new set of instructions to the AI according to at least one of conversation cues of an interview and instructions of a service provider.

According to some embodiments of the invention, the conversation is between a subject and an avatar and the conversation cues are from the subject, and the service provider is a caretaker.

According to some embodiments of the invention, the conversation cue is a name selected from a group consisting of: disease type, disease name, symptom, caregiver name, type of treatment, test, procedure names, or a combination thereof.

According to some embodiments of the invention, the specific prompt files are organized in a hierarchical library arranged according to subspeciality, caregiver name, disease type, disease name, symptoms, treatment, tests, procedure names, or a combination thereof.

According to some embodiments of the invention, a library of the plurality of libraries includes ques and responses from recorded conversations of the service provider and each set of instructions is created from at least one of a question, a remark and an answer of the service provider regarding specific cues during the conversations.

According to some embodiments of the invention, a specific library of the plurality of libraries includes specific conversation cues from an existing validated database of symptoms and responses for a caretaker interview with a subject having a condition relating to the conversation cues.

According to some embodiments of the invention, the specific library is customized for a specific caretaker and includes cues from recorded conversations of the specific caretaker.

According to some embodiments of the invention, the specific library is changed in response to a conversating of the service provider with the avatar.

According to some embodiments of the invention, the specific library is customized for a specific caretaker and includes cues from recorded conversations of the specific caretaker and wherein the validated database is based accepted on medical guidelines.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1: A block diagram illustrating a caregiver avatar system, in accordance with some embodiments of the current invention.

FIG. 2: A block diagram illustrating a central processing module for a caregiver avatar system, in accordance with some embodiments of the current invention.

FIG. 3: A block diagram illustrating high level control of caregiver avatar system, in accordance with some embodiments of the current invention.

FIG. 4: A block diagram illustrating an artificial intelligence (AI) module of a caregiver avatar system, in accordance with some embodiments of the current invention.

FIG. 5: A schematic illustration of use of a caregiver avatar system, in accordance with some embodiments of the current invention.

FIG. 6: Schematic diagram illustrating a patient facing interface, in accordance with some embodiments.

FIG. 7: Schematic diagram illustrating a patient facing interface, in accordance with some embodiments.

FIG. 8: Schematic diagram illustrating a caregiver facing interface, in accordance with some embodiments.

FIG. 9: Schematic diagram illustrating a caregiver multi-view interface, in accordance with some embodiments of the current invention.

FIG. 10: A flow diagram of a method of use of a caregiver-patient interface, in accordance with some embodiments.

FIG. 11: A flow diagram of a method of use of a caregiver-patient interface, in accordance with some embodiments.

### DESCRIPTION OF SPECIFIC EMBODIMENTS OF THE INVENTION

The present invention, in some embodiments thereof, relates to a caregiver avatar system and, more particularly, but not exclusively, to enhance healthcare delivery and/or medical communication

### Overview

An aspect of some embodiments of the current invention relates to a caregiver avatar method and system is disclosed and described. Optionally, the method and system may be useful in medical communications. Optionally, the method and system may provide a patient with medical advice. Optionally, the method and system may enhance healthcare delivery and/or increase efficiency of medical practices. Optionally, the method and system may assist a caregiver in evaluating patients. Optionally, the method and system may be configured to facilitate pre-visit information gathering, rapid and accurate diagnosis, treatment follow-up, scheduling patient visits, scheduling patient tests, providing remote tests, follow-ups, issuing prescriptions, renewing prescriptions, ordering transportation, etc. Optionally, the system and method may streamline administrative tasks. Optionally, the system may be configured to reduce the burden on caregivers, facilitating the dedication of more time to patient care. For example, in response to a diagnosis from the caretaker and/or the Avatar system, the Avatar system may order a medicine and/or a piece of medical equipment (e.g., an oxygen supply and/or a blood oxygen meter and/or

In some embodiments, the Avatar system may schedule of a secondary caretaker, for example, a nurse practitioner, to visit a patient receiving home care following heart surgery. During her assessment of the patient's medical condition, for example, taking note of his vital signs, medication regimen, and overall well-being. The Avatar may recognize critical factors for facilitating the patient's safety and/or comfort. For example, the Avatar with approval of a primary caretaker (e.g., a doctor) and/or the primary caretaker on his own may prescribe life-saving medical supplies to support ongoing care.

In some embodiments, in response to the prescription, the Avatar coordinates with a home health agency to order the necessary supplies. The Avatar optionally, reviews the prescription details, checking that the quantities and specifications matched patient needs. Additionally or alternatively, the Avatar may arrange for the supplies to be delivered directly to the patient's home and/or an institution housing the patient. The Avatar may check that the supplies are available when needed.

In some embodiments, delivery of supplies, the Avatar may supply on-line training and/or dispatch a trained medical technician to provide education and/or training use if the supplies. For example, the training may include proper procedures for administering medication, operating medical devices, and/or performing emergency procedures. The instructions may include safety instructions and/or allow questions and respond with answers.

Optionally, the Avatar monitors the condition of the patient, the supplies, the staff and/or the patient's environment and/or provides ongoing support. The Avatar may optionally perform follow-up monitoring and/or arrange for caretaker visits in response to treatment and/or to address issues that arise. Optionally, in cooperation with various caretakers and/or with approval of a primary caretaker the Avatar may make adjustments a care plan.

According to some embodiments, the system may include various components, such as a visual display for the patient and/or caregiver, a computing unit, a software module controlling a virtual avatar, and a large general language model artificial intelligence (AI). Preferably, these components may be integrated to provide a highly efficient and/or personalized patient evaluation experience and/or increase efficiency of use of medical resources.

According to some embodiments, a system may include a patient app. Optionally, the patient app may feature an interactive avatar. Optionally, the avatar may be linked to an AI and/or one or more databases. Optionally, the avatar may engage in a conversation with the patient. Optionally, the conversation may be verbal (e.g., video and/or audio) and/or haptic (e.g., chat, etc.). Optionally, the conversation may be verbal, but include subtitles e.g., for better understanding. Optionally, the conversation may focus on a particular health issue. For example, the conversation may begin by the avatar opening the conversation with warm up conversation, the conversation may include asking the patient relevant questions (e.g., medical history, current medical condition, emotional state, etc.), and based on the patient's responses and/or the patient's medical record and/or other factors (e.g., news about prevalent issues (epidemics, common stresses and/or social issues and/or medical issues in the patient's community), environmental issues in the patient's location, known issues in the patient's life), the AI through the avatar may intelligently generate additional inquiries, ensuring a thorough evaluation. At times the system may include filler conversation to engage the patient and give a caretaker time to take into account and/or react to notifications.

According to some embodiments, during the conversation and/or once the conversation is complete, the system may generate a summary of the results and/or send information (e.g., notifications [e.g., warnings, progress reports, suggested interventions, questions on how to proceed, information that may be significant from the medical record and/or other sources] and/or the summary) to a caregiver. Optionally, the system may provide concluding remarks. Optionally, the concluding remarks may include possible diagnoses and/or recommended actions, warnings about possible complications. Optionally, the conversation may be stored in a health care database. Optionally, the summary and/or concluding remarks may be presented to a caregiver e.g., by email, through an app, etc. Optionally, the conversation and/or medical history of the patient may be accessible to the caregiver. Optionally, the caregiver may review and/or evaluate coverage of the medical issue by the system's questions. Optionally, the avatar's questions may guide the patient through the conversation, leading to a comprehensive understanding of the patient's condition.

According to some embodiments, the avatar's questions may be guided by a caregiver. Optionally, a caregiver may evaluate if the questions asked provided proper coverage of the issue by the avatar's questions and conclusions. Optionally, a caregiver may instruct the avatar to ask additional questions, address a particular issue, change the diagnosis, or adjust the proposed treatment and/or recommendations for further testing, such as, blood tests, imaging tests, functional tests, drug reaction, therapies, etc. Optionally, the system may evaluate imaging tests in context of the other data from the patients. Optionally, the system may identify specific skin disorders, e.g., lesions. Optionally, the system may suggest a diagnosis. Optionally, the system may evaluate complex data recorded at multiple time points, such as but not limited to: HRV (Heart Rate Variability) data, multiple blood pressure measuring, oxygen saturation, EEG (Electroencephalogram) recording, ECG (Electrocardiogram) recording, EMG (Electromyograph) recording, ultrasound recording, photoplethysmography recording, breathing gases recording, pulmonary gases recording, pH recording, electrical evoked response recording, etc. Optionally, the system may interpret the data in the context of the other data, and/or identify abnormities and/or suggest a diagnosis. Optionally, the caregiver may review and/or evaluate the system's conclusions. Optionally, the caregiver may instruct the avatar to ask additional questions, and/or schedule tests, and/or schedule procedures, and/or follow ups, and/or prescribe medication to the patient.

According to some embodiments, the caregiver's decision-making process may be enhanced by use of the system. Optionally, the system may integrate conversation-based information with data from the patient's electronic medical records, data from the Internet (e.g., local environmental conditions, issues in the patient's community, issues in the patient's genetic cohorts. Optionally, the patient's electronic medical records may be accessed by the system remotely. Optionally, the patient may be monitored remotely. Optionally, the system may submit conclusions to a caregiver remotely e.g., a specialist, etc. Optionally, the system may submit the conclusion to more than one caregiver for interdisciplinary consultation. Optionally, the system may submit the conclusions together with the data from the electronic medical records to the caregiver. Optionally, integration of data may facilitate a holistic approach to care of the patient. Optionally, such a holistic approach may provide the caregiver with a comprehensive overview of the patient's health status, thereby enabling the caregiver to make informed decisions and/or provide appropriate care. In some embodiments, the system will access other patient data such as scheduling data from his phone and/or health information from a smart watch and/or use information from a cell phone.

According to some embodiments, the system may play a role in completing and/or continuously updating the electronic medical records (EMR) of patients e.g., in medical institutions. Optionally, the system may capture and/or integrate information from patient-caregiver discussions, consultations with other caregivers, test results and/or interpretations thereof, caregiver's summaries, prescriptions, scheduled follow-up visits, etc. Optionally, the system facilitates real-time documentation. Optionally, the system may complete and/or continuously update the electronic medical records of the patient in various medical institutions according to the discussions between the patient and caregiver, consultations, test results and analysis thereof, caregiver summaries, prescriptions, scheduled follow up visits, etc. Optionally, the system may access different electronic medical record modules in a verbal and/or written manner, and/or analyze and/or evaluate data and/or introduce data in the electronic medical records of the patient. Optionally, the system may improve the accuracy and/or accessibility of patient records. Optionally, the system may enhance continuity of care. Optionally, the system may take care of actions relating to billing.

According to some embodiments, the system may access different electronic health record (EHR) modules through verbal and/or written conversation. Optionally, the system may analyze and/or evaluate data. Optionally, the system may introduce information into the electronic health record (EHR). Optionally, integration of data may enable efficient data management and/or contribute to a comprehensive patient record.

According to some embodiments, the system may offer flexibility in presenting the information to the caregiver. Optionally, the information may be presented to the caregiver in a synchronous and/or asynchronous manner, e.g., depending on the need. Optionally, the caregiver may access and/or evaluate the summary and/or the patient's electronic medical records at their convenience. Optionally, this may improve a caregiver's time usage.

According to some embodiments, the system may include a collaborative feature. Optionally, the collaborative feature may empower the caregiver by facilitating direct interaction with the patient through the avatar. Optionally, the caregiver may provide instructions for additional questions, schedule tests, procedures, follow-ups, prescribe medication to the patient, etc. Optionally, the collaborative feature may enhance the accuracy and/or efficiency of the evaluation process. Optionally, the caregiver may guide the conversation and/or provide specific guidance based on their expertise. In some embodiments diagnoses, inquiries, tests and/or recommendations may be generated by the system that require caretaker approval before being presented to a patient. For example, certain tests may have an element of danger and/or a likelihood of putting stress on the patient and/or have social connotations that need to be evaluated carefully before action.

According to some embodiments, the system may extend its capabilities to face-to-face physical visits and/or telemedicine visits between the caregiver and the patient. Optionally, the system may record and/or summarize the encounter. Optionally, the system may capture important details of the visit that may be added to the patient's medical records. Optionally, the system may provide a comprehensive account of the visit. Optionally, the system may offer suggestions for treatment and/or additional tests based on the information discussed during the visit. Optionally, the system may be configured to support the caregiver's decision-making process.

According to some embodiments, the system may be present at the visit in the form of the avatar. Optionally, during the visit, the avatar may be visible to caregiver and/or patient. Optionally, the avatar may provide reassurance to the patient. Optionally, the avatar may provide a sense of continuity to the patient, e.g., provide a familiar face from pre-visit and/or administrative interactions. Optionally, the system may schedule tests, or procedures in real time. Optionally, the caregiver's remarks and diagnoses, medication, additional tests, etc. may be introduced into the electronic medical record in real-time and/or after the visit by the system. According to some embodiments, the system may be present at the visit in the form of the avatar which may be visible to the caregiver exclusively. Optionally, the presence of the avatar may facilitate confidentiality and/or a focused interaction with the patient. Optionally, the system may present up-to-date information about new medication interactions, etc. Optionally, the patient may feel at ease, knowing that their health information is being shared with the caregiver alone.

According to some embodiments, the avatar may be powered by a general language model AI. Optionally, the avatar may provide a human-like interaction experience. Optionally, the avatar may be controlled and/or animated by a module. Optionally, the avatar itself may be a realistic 3D animation, the image of a real person, e.g., a specific caregiver. Optionally, the module may be configured to enable the avatar to display emotions, compassion, understanding, a sense of humor, etc. Optionally, the module may be configured to assess a patient's level of understanding and/or to provide information and/or ask questions in language appropriate to the patient's level of understanding. Optionally, the module may be configured to enhance the patient's engagement and/or connection with the avatar, e.g., fostering trust and/or openness during the evaluation process.

According to some embodiments, the avatar's appearance may be customizable, e.g., to resemble a real person, such as the caregiver or another individual. Optionally, the avatar may be customized to include the caregiver's voice, facial expressions, lips, gestures, etc. Optionally, the avatar's voice, facial expressions, lip movements, gestures, etc. may be realistically animated. Optionally, the avatar's lifelike representation may enhance the patient's connection with the avatar, creating a more immersive and engaging experience. Optionally, the system may provide customization options to the caregiver. Optionally, the caregiver may select specific emotions, attitudes, tone of voice, etc. for the avatar. Optionally, the caregiver may align the avatar's characteristics with their desired approach and/or communication style.

According to some embodiments, the system may incorporate advanced capabilities for emotional assessment. Optionally, the control module of the system may evaluate the patient's emotions e.g., based on their language, facial expressions, voice, gestures, etc. Optionally, the avatar may react and/or respond to a patient's emotions appropriately, e.g., by displaying empathy, concern, reassurance, etc. Optionally, the system's emotional intelligence may enhance the patient's well-being. Optionally, the system's emotional intelligence may facilitate a supportive and/or comforting environment.

According to some embodiments, the system may evaluate the patient's level of understanding, e.g., by analyzing their language, facial expressions, voice, gestures, etc. Optionally, the system may adjust the complexity of its answers and/or explanations to match the patient's comprehension level. Optionally, the system may promote effective communication and/or understanding. Optionally, the system may display visual aids, such as sketches, animations of tests and procedures, etc. Optionally, visual aids may enhance patient understanding and/or engagement.

According to some embodiments, the avatar may support the informed consent process, e.g., by presenting information in a conversational manner, using language that patients can understand, showing visual aids, providing a sympathetic ear, etc. Optionally, the avatar may facilitate that patients comprehend the details of tests and/or procedures, check their understanding, and/or provides a summary for signing by the patient and/or caregiver.

According to some embodiments, the avatar may serve as a companion to the patient, providing frequent conversation and/or support during their healthcare journey, e.g., during their stay in a medical institution, when they require emotional support and/or further information, regardless of the time of day. Optionally, the avatar may assist in alleviating patient apprehension, explaining upcoming tests and/or procedures, outline expected results and/or potential side effects, and/or offer guidance on managing them. Optionally, this companion-like interaction may enhance the patient's experience and/or promote their well-being and/or healing. Optionally, the system may convey information learned in communicating with this patent to the caregiver (e.g., this patient is very sensitive to fear triggers and/or questions should be asked in a way that does not imply suspicion of a frightening medical condition).

According to some embodiments, the system may include continuous training of the AI. Optionally, continuous training may be an integral part of the system. Optionally, the caregiver may train the system in a conversational manner. Optionally, the caregiver may provide specific instructions for asking specific questions on particular occasions, and/or to give detailed explanations including showing sketches and/or animations of tests or procedures. Optionally, the module controlling the large language model can be trained individually for each clinical specialty.

According to some embodiments, the system may be versatile and/or adaptable. Optionally, the system may facilitate specialty-specific training and/or instructions. Optionally, the caregiver's training instructions may be saved for their personal use, use by a particular clinic, and/or utilized by an avatar system dedicated to a particular medical specialty. Optionally, training may facilitate a tailored approach to patient evaluations.

According to some embodiments, the system may recognize the importance of language preferences and/or patient understanding. Optionally, the avatar's language may be changed according to the patient's preference (e.g., Spanish, English, German, Chinese, etc.). Optionally, the avatar's language may be changed according to the patient's level of understanding. Optionally, the system may facilitate effective communication and/or promote patient-centered care.

According to some embodiments, the system may incorporate various modules for advanced evaluation. Optionally, the system may analyze different patient features, such as facial expressions, symmetry movements, color, skin turgor, voice pattern, sentence articulation, coherence, speaking pattern, timber, breathing sounds, gestures, gait, posture, general tidiness, photoplethysmography, etc. In some embodiments, the system may be sensitive to environmental factors (e.g., if the patient is speaking from his home, what are the conditions of the room, is the patient sitting, walking, involved in another activity etc.). Optionally, these evaluations may assist in identifying signs and/or indications of diseases, changes in the severity of chronic illnesses, etc. Optionally, these modules may enable early detection and/or continuous monitoring.

According to some embodiments, the system may access remote sensors e.g., on a patient's personal computing device, dedicated sensors, etc. Optionally, the system may request the performance of specific tests and/or activities to assess a patient's condition. Optionally, the system may send the patient for testing, e.g., blood tests, ECG, etc. Optionally, the system may send a test to a patient, e.g., a home test such as Corona, urine collection, etc. Optionally, the system may provide the patient with one or more sensors for home testing, e.g., glucose test, blood pressure measurement, heart rate, etc. Optionally, the system may send a test to a patient in a clinic, e.g., a nurse with instructions to perform a particular test and/or to take the patient to a dedicated location to perform a specific test, e.g., treadmill test, MRI (Magnetic Resonance Imaging), etc.

According to some embodiments, the system, through the avatar may instruct the patient to perform certain tests and/or look for specific signs. Optionally, the tests may include performing manual tasks e.g., for assessing force or dexterity; walking for some distance; checking for equilibrium; breathing; saying a sentence; discerning letters at a distance; checking the sense of smell; checking for sensation in hands, feet, or face; etc. Optionally, the tests may involve measuring weight, height, temperature, checking blood pressure, pulse rate, heart rate variability (HRV), checking saturation, a home blood test, a home sputum test, performing an ECG, performing a breathing gases test, checking ocular pressure, using an otoscope to look into one's ear. Optionally, the signs may include taking a photograph and/or video of skin discoloration, swelling of body, eyelids, abdomen, or feet, looking in the mouth, or oropharynx, or tongue, taking a middle ear image, etc.

According to some embodiments, the system may include modules for remote physiological measurements. Optionally, the system may be configured to monitor and/or evaluate the patient by photoplethysmography (PPG) of the face. Optionally, the system may evaluate pulse rate variability, oxygen saturation, breathing rate, blood pressure, blood glucose levels, etc. Optionally, the system may use non-invasive measurements to provide real-time assessment of vital signs during the conversation. Optionally, real-time assessment of vital signs may contribute to a comprehensive evaluation of a patient's condition.

According to some embodiments, the system's capabilities may extend to measuring physiological parameters through contact PPG of the patient's finger e.g., on a telephone, remote sensor, etc. Optionally, PPG measurement may facilitate additional vital sign measurements, such as temperature, stress level, caloric intake, and extended monitoring of blood glucose levels. Optionally, PPG may enable the evaluation of electrocardiogram (ECG or ECG), electroencephalogram (EEG), electromyogram (EMG) and electrooculogram (EOG), respiration gas levels, and skin sympathetic activity, enhancing the scope of health assessment.

According to some embodiments, the system may be used to identify, treat and/or monitor a wide range of illnesses. Optionally, the versatility of the system may facilitate comprehensive evaluations across multiple medical domains. Non-limiting examples of diseases which may be identified, treated, and/or monitored by the system may include: chronic heart failure and heart diseases, Chronic Obstructive Pulmonary Disease (COPD), asthma, chronic liver disease, ischemic heart disease, stroke, neurologic disease, chronic kidney disease, autoimmune diseases, orthopedic diseases, depression, anxiety disorders, addictions, compulsive disorders, and other psychiatric problems.

According to some embodiments, the system may be tailored for a specific healthcare sector. According to some embodiments, the system may not replace the caregiver. Optionally, the system may enhance healthcare delivery and improve medical practices without replacing the caregiver.

According to some embodiments, the system may improve the usage of multiple caregivers' time e.g., by considering their availability and directing patients to the available caregivers at the end of the conversation. Optionally, intelligent scheduling by the system may facilitate efficient allocation of resources, and/or reduce patient wait times and/or improve the utilization of healthcare professionals and/or coordinating preparation times and/or follow up and/or preferred times for tests or treatments (e.g., for children certain tests are preferred in the morning where the child may be more alert and/or cooperative) and/or preferred locations. For example, the system may include information about transport (traffic, public transport) and patient locations.

According to some embodiments, the system may expedite the treatment process by evaluating imaging and other test results, e.g., providing immediate preliminary conclusions and/or responses. Optionally, rapid feedback to patients and/or caregivers may enable timely decision-making and/or enhance the overall efficiency of healthcare delivery.

According to some embodiments, the system may assist in determining the best sequence and/or timing of multiple tests, e.g., by considering facility availability, expected test or procedure durations, personnel schedules, patient schedule, physical distance between tests, expected wait times, recovery times, preparations etc. Optionally, the system may facilitate transportation within medical institutions, e.g., by scheduling it for patients. Optionally, the system may facilitate smooth and/or efficient movement within the healthcare facility, and/or reduce logistical challenges for patients and/or caregivers. Optionally, such improvements may streamline the patient's diagnostic journey, and/or reduce delays, and/or improve the overall patient experience.

According to some embodiments, the system's capabilities may extend beyond patient evaluations. Optionally, the system may evaluate the time usage, and/or revenue generation, and/or efficacy of different caregivers and sections within a medical institution. Optionally, data-driven evaluation may aid in improving resource allocation and/or improving overall healthcare outcomes.

According to some embodiments, the system may assess the efficacy of caregivers in treating patients, e.g., by taking into account factors such as time usage, resource usage, side effects, complications, patient satisfaction, etc. Optionally, the evaluation may promote continuous improvement and/or facilitate delivery of high-quality care.

According to some embodiments, the described system and method may leverage advanced technologies, such as virtual avatars, language models AI, and integrated modules, for assessment and/or documentation. Optionally, the system may enhance the efficiency, accuracy, and/or personalization of patient care. Optionally, the system may benefit patients and/or caregivers in a wide range of medical specialties and/or healthcare settings.

According to some embodiments, the system may seamlessly computing capabilities with immersive visual interfaces to create a virtual counterpart of a real-life caregiver. Optionally, the system may deliver a patient experience that is virtually indistinguishable from face-to-face interactions. Optionally, the system and method may integrate healthcare and artificial intelligence. Optionally, the system and method may bridge the gap between virtual and real-world healthcare experiences. Optionally, the system and method may replicate the actions, communication style, and/or decision-making processes of each caregiver. Optionally, the system and method may create a personal caregiver-virtual avatar which may operate as an extension of the healthcare provider. Optionally, the avatar's capabilities may be achieved through the synergistic operation of various components and/or modules. Optionally, each component and/or module may be designed to enhance patient-caregiver interactions.

According to some embodiments, the system may include a user interface to handle semi-automated caregiver-patient interactions. Optionally, the system may include two or more caregiver interfaces. Optionally, the caregiver interfaces may be a direct chat interface e.g., the caregiver may see and/or interrupt in real time a chat between the patient and the avatar. Optionally, the caregiver interfaces may include a control interface.

According to some embodiments, the caregiver may give general instructions through the interface (e.g., ask about symptoms connected to stroke, ask about family history of heart disease, do tests of awareness/stamina, check up on drug use compliance). Optionally, caregiver instructions may be simple commands which may trigger complex avatar actions. For example, the system may check for signs of poor compliance in the condition of the patent and/or by collecting ancillary data such as purchase receipts and/or via collecting data from an ancillary device. For example, the system may collect data from a smart refrigerator as to whether the right bin was opened at the right time and/or the system may collect data from a phone GPS as to whether the patient was in a place of treatment and/or the system may collect data from an injection device and/or a testing device (e.g., a glucose checking device) the system may collect data from a smartwatch that indicates proper follow up and/or compliance.

According to some embodiments, the caregiver may check up on care quality. For example, the system may check for signs of poor care in the condition of the patent and/or by collecting ancillary data such as purchase receipts and/or via collecting data from an ancillary device. For example, the system may collect data from a smart refrigerator as to whether the right bin was opened at the right time and/or the system may collect data from a phone sensor as to whether the patient was moved or left without physical care or physical therapy. For example, the system may monitor for signs of improvement after an intervention and/or for early signs of distress before proper diagnosis.

According to some embodiments, the caregiver may check up on improper medicine use. For example, the system may check for signs that the patient is not using a prescribed medicine (e.g., physiological changes expected from the use, accessing a refrigerator at an expected time (and/or possibly selling it off illegally) and/or a history of unusual prescriptions to a patient and/or a history of unusual prescriptions by a doctor.

According to some embodiments, the caregiver may receive information through the interface, e.g., structured summaries of various information what symptoms are present (include those mentioned by the patient, apparent in the patient's appearance [such as, appearance of skin (e.g., pale, presence of patches, sweat, discoloration, color of lips, etc.), and/or appearance of eyes (e.g., dilation, synchronization, eye contact, twitching, alignment, reaction to changes of light, equality, etc.) and/or behavior (e.g., signs of anxiety, confusion, depression, tremors, tics, etc.), and/or speech (e.g., word use, voice, hesitation, rate, blocking, circumlocution, slurring mispronunciation, circumstantial speech, wording errors, etc.), and/or results of tests, and/or medical history (e.g., what treatments have been used, what was the response, etc.), and/or general information (e.g., access medical databases, information over the Internet, proprietary databases, etc.).

According to some embodiments, the interface may facilitate simultaneous interactions with multiple patients.

According to some embodiments, the interface may make recommendations to the caregiver (e.g., independent, monitor caregiver instructions, warn when the caregiver may be missing something, acting not in accordance with accepted practice, etc.).

According to some embodiments, the interface may record sessions for later use (e.g., in-depth analysis for improved diagnosis and/or treatment, use when necessary for insurance and/or legal situations, and/or review of quality of care, etc.).

According to some embodiments, the system may include a chat interface for a patient. Optionally, the patient may interact with a caregiver and/or bot (e.g., avatar) through the interface. Optionally, the interactions between the caregiver and the avatar may be configured to appear the same e.g., so that it is not immediately apparent to the patient with whom they are interacting. Alternatively, or additionally, interaction with the bot and the doctor may be differentiated.

In some embodiments, a caretaker (e.g., the doctor) may take over a session. For example, the caretaker may communicate directly with the patient. Optionally, the caretaker may silence Avatar communication with the patient so that patient receives input only from the caretaker. Optionally, the caretaker may silence Avatar communication with the caretaker e.g., helping the caretake direct more concentrated care to the patient.

According to some embodiments, the avatar may be configured to appear realistically like the caregiver, e.g., to encourage the patient to interact freely. Optionally, the avatar may appear in a video call with the appropriate facial expression and/or voice intonation, etc. Optionally, the avatar may have mannerisms and/or speech patterns and/or voice intonations, etc., which may be customized to mimic those of the specific caregiver. Optionally, the avatar may have an independent and/or semi-independent mode. Optionally, the avatar may be accessible to the patient at any time, e.g., the patient may contact the avatar 24/7 and get information and/or report changes in their condition.

According to some embodiments, the system may collect data and/or send a summary to a caregiver (e.g., doctor, nurse, etc.). Optionally, the data collection may include active interviewing and/or questioning of the patent. Optionally, the data collection may include administration of tests, e.g., active tests such as, look at camera and shut/turn on lights, climb flight of stairs and report back, eat an orange and report how you feel, check blood sugar, etc. Optionally, the data collection may include collection of information from patient associated devices (e.g., smart watch, cell phone, cell phone sensors, etc.). Optionally, the data collection may include accessing the patient's medical records, optionally including records from multiple doctors and/or institutions.

In some embodiments, the Avatar may access (e.g., store and/or retrieve data) of a secure medical records system. For example, the secure medical records system may be designed to protect sensitive patient information from unauthorized access, tampering, and/or disclosure. Optionally the secure medical record system employs robust access control mechanisms to restrict access to patient records only to authorized entities, such as healthcare providers directly involved in the patient's care. In some embodiments, access privileges are granted based on role-based access control (RBAC). Optionally, access is gained with multi-factor authentication (MFA) enhancing security by requiring users to provide multiple forms of verification. Patient data is optionally encrypted in transit and/or at rest. For example, a strong encryption algorithm may be used to facilitate confidentiality and/or integrity. Optionally, the Avatar and/or another entity performs audit trails records of access and/or activities related to patient records, stored securely to prevent unauthorized modification. Optionally, the Avatar and/or another entity performs regular backups and/or a comprehensive disaster recovery plan. For example, the plan may inhibit data loss in case of hardware failures or disasters. Data minimization principles are optionally followed to reduce the stored information. Optionally there are retention policies governing data storage and deletion. In some embodiments, secure communication protocols are used to facilitate encrypted data transmission. Optionally, the Avatar and/or another entity performs security audits and/or compliance checks that assess, for example, the system's security posture and facilitate compliance with relevant regulations such as HIPAA or GDPR.

According to some embodiments, the system may alert the caregiver and/or emergency services to possibly dangerous situations. According to some embodiments, the system may reach out to a patient to facilitate and/or monitor their condition and/or their compliance with treatment regime.

According to some embodiments, the system may include instructions limiting and/or controlling the AI. Optionally, the AI may be prevented from taking certain actions, e.g., prescribing treatments without approval of a caregiver, suggesting tests and/or actions that may be dangerous, stressful, objectionable to a particular patient, etc.

According to some embodiments, the system may facilitate dynamic injection of data and/or instruction to an AI system. Optionally, a lot of data and/or functions may not be handled simultaneously by the AI routine. Optionally, the system may include various databases (e.g., libraries) for different patient conditions (e.g., emergencies, heart conditions, infections, mental problems, etc.). Optionally, the AI may be given a specific set of instructions which may change according to the progress of the session and/or instructions of the caregiver. Optionally, the system may include a very large library of functionality (e.g., larger than the AI is capable of handling at a given moment). Optionally, information from the libraries may be injected into the AI instructions in accordance with need and/or current situation. Optionally, the patient's symptoms may be classified in a hierarchal library (e.g., includes possible suspected conditions, groups of symptoms, queries, etc.). Optionally, test results and/or patient inquiries may be classified in a hierarchal library. Optionally, each library may be configured for a particular caregiver (e.g., individual behavior, mannerisms etc. Optionally, the data of the conversation may be saved in a structured way creating an electronic record system. Optionally, the electronic record system may be continually expanded with additional data from each patient and from additional patients.

Some embodiments may relate to a method for evaluating patients comprising: a virtual avatar which may converse with a patient regarding a particular health issue, wherein the patient's answer to the avatar's question may leads to additional relevant questions from the avatar, and wherein a summary of the conversation may be presented to a supervising caregiver, and wherein the caregiver may review the summary to evaluate coverage of the issue by the avatar questions and regarding its conclusions included in the summary.

According to some embodiments, the supervising caregiver may instruct the avatar to ask additional questions. Optionally, the supervising caregiver may accept and/or change the diagnosis suggested by the system. Optionally, the supervising caregiver may accept and/or change the actions suggested by the system. Optionally, the actions suggested by the system may include blood tests, imaging tests, functional tests, drug reaction, therapies, etc. Optionally, supervision by the caregiver may be synchronous with the avatar conversation. Optionally, supervision by the caregiver may be asynchronous with the avatar conversation.

According to some embodiments, the system may include a processor, a computer interface, and virtual reality software based on decision making software and supported by generative AI, and a database for evaluating patients. Optionally, the system may include a virtual avatar which may interact with a patient regarding a particular health issue. Optionally, the system may include a module to evaluate during the conversation different patients features chosen from a group of: the patients face features, symmetry movements, color, expression, skin turgor, puffiness, sweating, voice pattern, sentences articulation a coherence, speaking pattern and timber, breathing sounds, gestures, gait posture, general tidiness, regarding signs or indications of diseases or to determine change of severity of chronic illnesses. Optionally, the system may include a module to measure during conversation of remote PPG of the face for evaluating pulse rate variability, O2 saturation, breathing rate, blood pressure, blood glucose levels, etc. Optionally, the system may include a module to measure during the conversation through contact PPG of the patient finger with a telephone or other remote sensor. Optionally, the module may evaluate pulse rate variability, O2 saturation, breathing rate, blood pressure, temperature, stress level, caloric intake or blood glucose levels, ECG, EMG, EEG, respiration gas levels, skin sympathetic activity, etc.

According to some embodiments, the system may include a visual display for a patient and another for the caregiver, a computing unit, a software module controlling a virtual avatar, a module controlling a large general language model AI, a patient app including a patient display interface which includes the avatar.

According to some embodiments, the system may include a computing unit, a visual patient interface and a caregiver visual interface, a writable memory unit and a subsystem for generating and controlling a caregiver avatar. Optionally, the avatar may act in a similar manner as a specific caregiver. Optionally, the avatar may ask similar questions and/or give similar answers to what a particular caregiver would have given under similar circumstances. Optionally, a module may generate a realistic and animated caregiver avatar having the same facial features, voice and mannerisms as the particular caregiver e.g., when the avatar converses with a patient.

According to some embodiments, the subsystem may include a constant module for tailoring the answers and/or questions generated by a large language model (LLM). Optionally, the system may include a specific constant module for each different medical subspeciality. Optionally, the constant module may be developed as prompts controlling the activity of the LLM. Optionally, the LLM may be validated by using test cases and/or real caregiver-patient conversations.

According to some embodiments, the subsystem may include a conditional module. Optionally, the conditional module may select and/or use a specific prompt file for controlling the LLM answers and questions of a particular caregiver avatar at predetermined conversation cues. Optionally, a predetermined conversation cue may be a name selected from a group including: disease type, symptom, type of treatment, tests, procedure names, etc. Optionally, the specific prompt files may be organized in a hierarchical library organized according to subspeciality, caregiver name, disease type, symptoms, treatment, tests, procedures, etc.

According to some embodiments, the system may include an internal quality control AI based module. Optionally, the quality control module may compare conversational results of different caregivers in the same subspeciality. Optionally, the quality control module may relate to the same cues. Optionally, based on the quality control results from the LLM a particular caregiver in case of non-concordances may be prompted to adhere to a standardized action. Optionally, the quality control module may automatically adjust the conversation prompts of each caregiver in accordance with the latest instructions of the relevant medical association and/or medical guidelines.

According to some embodiments, a caregiver may instruct an avatar in the case of inadequate questions or answers. Optionally, such instructions be saved in the respective prompt files.

### Specific Embodiments

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Reference is now made to the figures.

FIG. 1 is a block diagram illustrating a caregiver avatar system, in accordance with some embodiments of the current invention. For example, in system 10, a virtual avatar 11 may interact with patient 12 through an interface, avatar 11 may converse with patient 12 to collect data. Optionally, the data collection may include active interviewing and/or questioning of the patent. Optionally, the data collection may include administration of tests, e.g., active tests such as, look at camera and shut/turn on lights, climb flight of stairs and report back, eat an orange and report how you feel, check blood sugar, etc. Optionally, data may be collected from one or more monitoring devices 14, such as patient associated devices (e.g., smart watch, cell phone, cell phone sensors, etc.). Optionally, the data collection may include accessing the patient's medical records, optionally including records from multiple doctors and/or institutions. Optionally, the patient's electronic medical records may be accessed by the system remotely. Optionally, the patient may be monitored remotely. Optionally, the system may submit conclusions to a caregiver 13 e.g., a specialist, etc. Optionally, the system may submit the conclusion to more than one caregiver for interdisciplinary consultation. Optionally, caregiver 13 may evaluate if the questions asked provided proper coverage of the issue by the avatar's questions and conclusions. Optionally, a caregiver 13 may instruct the avatar 11 to ask additional questions, change the diagnosis, adjust the proposed treatment and/or recommendations for further testing, such as, blood tests, imaging tests, functional tests, drug reaction, therapies, etc.

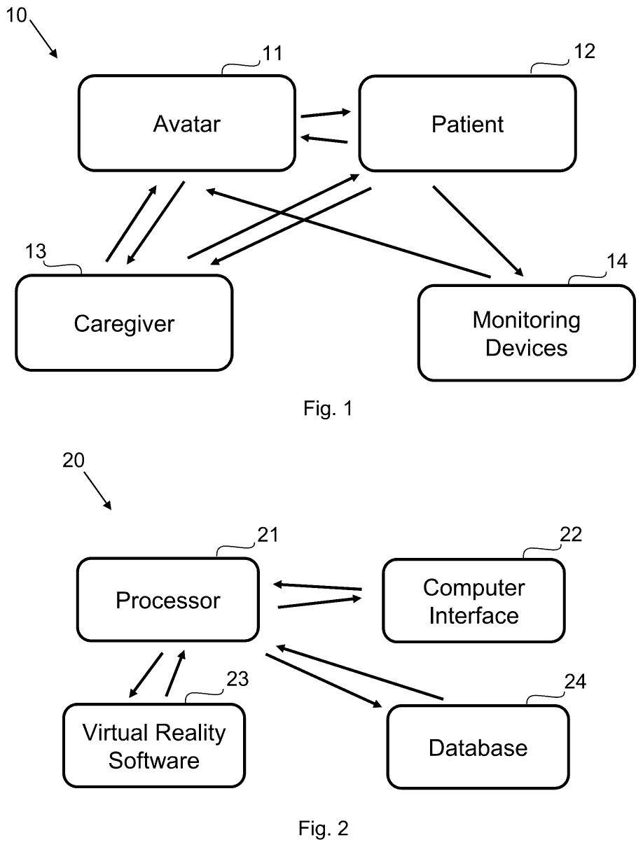

FIG. 2 is a block diagram illustrating a caregiver avatar system, in accordance with some embodiments of the current invention. For example, in system 20, a caregiver avatar system may comprise a processor 21, a computer interface 22, and virtual reality software based on decision making software, supported by generative AI 23, and a database 24 for evaluating patients with the help of a virtual avatar.

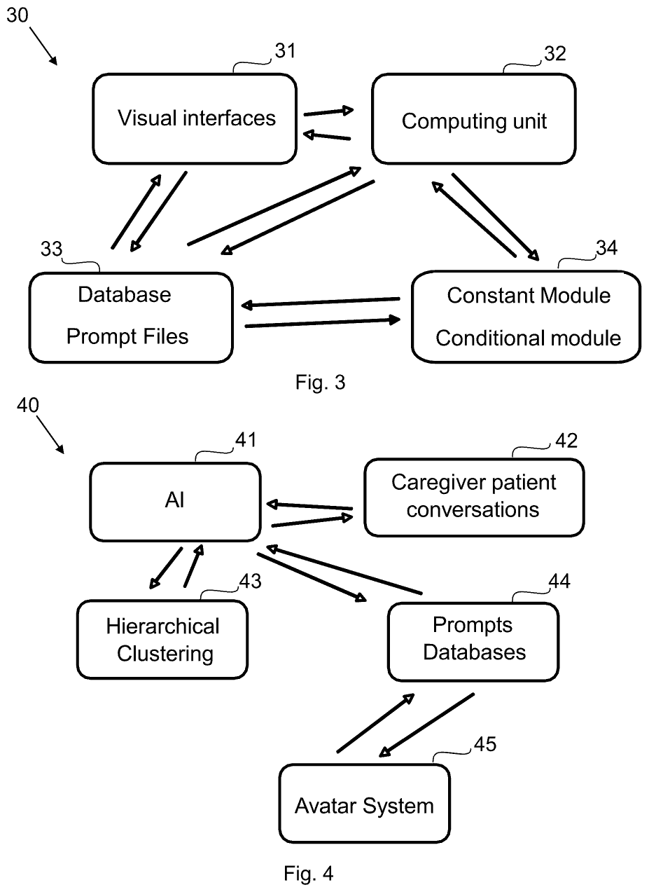

FIG. 3 is a block diagram illustrating a caregiver avatar system, in accordance with some embodiments of the current invention. For example, system 30 is anchored by a computing unit 32 that serves as its computational brain. This unit 32 may process large amounts of data in real-time. Optionally, interactions between patients and their virtual caregiver counterparts may therefore be seamless. Optionally, to facilitate effective communication, the system may offer visually intuitive interfaces 31 for patients and/or caregivers. Optionally, these interfaces may be designed to create an immersive and user-friendly experience, e.g., making interactions more engaging and informative. Optionally, the system may include a writable memory unit. Optionally, the writable memory unit may enable the storage and retrieval of information necessary for maintaining continuity in patient interactions. Optionally, the storage may be cloud-based. Optionally, the system includes a subsystem responsible for generating and/or controlling the virtual caregiver avatar.

According to some embodiments, the avatar may be configured to faithfully replicate a caregiver's actions and communication style. Optionally, the avatar may be programmed to ask questions and/or provide responses in a manner consistent with how the designated caregiver would behave in similar clinical scenarios. Optionally, a high level of accuracy and customization may be achieved through the integration of the Constant Module and Conditional Module 34. For example, certain instructions, training parameters and/or data may be constantly available (e.g., caregiver specific behaviors, emergency triggers and/or reactions). In some embodiments, the conditional module may be handled dynamically. Optionally, other instructions, training parameters and/or data may be injected into the active instructions and/or stored in for later use.

According to some embodiments, the Constant Module may tailor the avatar's behavior to match that of the real caregiver. Optionally, the Constant Module may fine-tune the responses generated by a Large Language Model (LLM) to facilitate that they align closely with the mannerisms and/or communication style of a designated caregiver in a specific subspecialty. Optionally, the system may create a virtual caregiver avatar that is both visually realistic and/or animated. Optionally, this avatar may capture not only the facial features and voice of the real caregiver but also replicates their mannerisms and gestures. Optionally, such customization may make the virtual experience lifelike and/or informative. Optionally, patient interactions with the avatar may therefore be immersive, e.g., instilling a sense of trust and familiarity.

According to some embodiments, recognizing the diversity within the field of medicine, the system may include specialized Constant Modules tailored for various subspecialties. Optionally, these modules may facilitate the virtual caregiver to adapt to the unique requirements and practices of different medical domains. For example, whether dealing with cardiology, neurology, or orthopedics, the system may customize the avatar's behavior to match the specific demands of each subspecialty.

According to some embodiments, the development of each Constant Module may be a rigorous process. Optionally, each Constant Module may be meticulously crafted to act as prompts 33 controlling the LLM's behavior. Optionally, these prompts may undergo extensive validation through real-world test cases and/or caregiver-patient conversations to facilitate accuracy and/or appropriateness. Optionally, validation and quality control may be pivotal in maintaining the highest standards of care.

According to some embodiments, to facilitate that interactions are contextually relevant, the system may incorporate a Conditional Module. Optionally, the Conditional Module may select and/or deploy specific prompt files 33 to control the LLM's responses and/or queries at precise moments during a conversation. Optionally, these moments may be determined by predetermined conversation cues, such as disease type, symptoms, treatment, tests, and procedure names, which guide the avatar's behavior.

According to some embodiments, a hierarchical library of prompt files may enhance the system's organization. Optionally, prompt files may be organized based on factors such as, subspecialty, caregiver name, disease type, symptoms, treatment, tests, and procedures. Optionally, this hierarchical structure may simplify access and customization, enabling efficient use of the system's capabilities.

According to some embodiments, to maintain a high standard of care, the system may include an internal quality control AI module. Optionally, this module may compare conversational outcomes among caregivers within the same subspecialty. Optionally, deviations from standardized practices may trigger alerts to the specific caregiver, ensuring that the system consistently delivers high-quality care. Optionally, the quality control module may automatically update the avatar's conversation style in line with the latest directives from relevant medical associations, guaranteeing compliance with best practices.

Optionally, caregiver-initiated customization may empower caregivers to actively participate in the avatar's refinement process. Optionally, the system may be configured to allow caregivers to train their avatar in a conversational intuitive way. Optionally, when questions and/or answers are deemed inadequate, caregivers may make adjustments in real time. Optionally, the changes may be saved in the corresponding prompt files. Optionally, this may contribute to ongoing improvements in the system's performance.

FIG. 4 is a block diagram describing a caregiver avatar system, in accordance with some embodiments of the current invention. For example, in system 40, the system generates a comprehensive hierarchical database of prompt files 44. This database is produced by training an AI module 41 on actual caregiver-patient conversations 42. It captures caregiver behavior for LLM-controlled responses and questions in specific clinical scenarios, resulting in cues-related prompt files. The database relies on hierarchical clustering 43 to categorize caregiver conversations, illustrations, or prompts based on subspecialty, caregiver name, disease type, symptoms, treatment, tests, and procedures. Categorization enhances precision and accessibility when accessing and customizing prompts.

According to some embodiments, the avatar system may be designed for healthcare. Optionally, the versatility of the avatar system may facilitate its application in various user scenarios. Optionally, the system may seamlessly adapt, replacing the caregiver with a first user and the patient with a second user, facilitating interactive experiences across different domains.

According to some embodiments, the avatar system and method may be extended beyond patient interactions. Optionally, the system may offer a dynamic approach to electronic medical records (EMR) creation, expansion, and management. Optionally, the system may revolutionize healthcare data management while maintaining strict compliance with Health Insurance Portability and Accountability Act (HIPAA) regulations.

According to some embodiments, the system may create and expand hierarchical databases with patient data, effectively building electronic medical records (EMR). Optionally, the system may create and expand hierarchical databases automatically. Optionally, the avatar 46 may be a pivotal tool in this process, facilitating capture of patient information, medical history, treatment plans, and more during conversations.

In some embodiments, an AI module may by supplied and/or modified dynamically. For example, instructions may be changed in reaction to a suspected condition of the patient and/or instructions from the caretaker and/or a request from the patient and/or an observed condition. For example, dynamic data may include some or all of customized instructions, AI routines based on different training data and/or fine training parameters. For example, customized instruction may include instructions or guidelines to the AI module to prioritize certain tasks, objectives, or behaviors over others; defining the goals of the AI system, specifying desired outcomes, and outlining constraints or requirements; for example, in natural language processing tasks, instructions may include guidelines for sentiment analysis, topic classification, or entity recognition. For example, the system may change AI routines to a new AI routine based on a different training data based on a response from a patient and/or an observation. For example, the new model may be based on different training data. For example, training data may include, data resulting from teaching the AI module to recognize patterns, make predictions, or perform tasks; training based on datasets that are relevant to the specific domain, application, or problem; training that encompass a wide range of scenarios, variations, and edge cases that are suspected to be relevant to a particular patient at a particular time; for example, if a patient interview arouses a suspicion of a certain condition the system may dynamically load images specific training and/or analyze existing medical images for diagnosing diseases and/or analyze the demeanor and/or behavior of the patient with the disease specific instructions. Fine-tuning parameters may include changing configurable parameters of the IA model to improve performance for specific a task that may be recognized during an interview. For example, parameters may be tweaked to improve accuracy, efficiency, or other desired metrics; parameters may include hyperparameters (e.g., learning rate, regularization strength) and/or model architecture choices; techniques such as grid search, random search, or Bayesian optimization may be used to explore the parameter space and improve the configuration. For example, in deep learning models, fine-tuning may involve adjusting the number of layers, the size of the layers, and/or the activation functions to improve performance on a particular task.

According to some embodiments, as patients interact with their virtual caregiver avatars, relevant data may be seamlessly integrated into the EMR, completing the patient's medical history. Optionally, such a process facilitates updating. For example, the system may amend EMRs to remain up-to-date and/or comprehensive and/or to offering a holistic view of the patient's health journey.

According to some embodiments, caregiver-Initiated customization may empower caregivers to tailor the avatar to their specific needs. Caregivers may create new categories within the EMR and structure data through conversational commands to the avatar. Optionally, this flexibility allows caregivers to adapt their EMR to unique patient cases, subspecialties, or evolving medical practices. Optionally, the ability to customize EMRs through conversations with the avatar streamlines data entry and management, making it a powerful tool for caregivers seeking efficiency and precision in their documentation.

According to some embodiments, data security and/or privacy, which are paramount in healthcare, may be facilitated by the system. Optionally, the system may store patient data in a HIPAA-compliant manner on secure cloud servers. Optionally, this may safeguard sensitive medical information. Optionally, the system may facilitate that the data remains confidential and/or accessible only to authorized personnel.

According to some embodiments, by leveraging cloud technology, the system may provide scalability and/or accessibility, e.g., enabling healthcare providers to access EMRs from anywhere while adhering to stringent privacy regulations.

According to some embodiments, healthcare providers may engage in conversations with the virtual caregiver avatar to access and/or retrieve summaries, reports, and various forms from the EMR. Optionally, the system may use a natural language interface. Optionally, this natural language interface may simplify the process of accessing critical patient information, and/or enhancing decision-making, and/or patient care.

According to some embodiments, the avatar system 45 may facilitate data management by integrating EMR creation, expansion, and customization into the patient-caregiver interaction. The avatar system 45 may facilitate that patient data is captured, organized, and stored in a secure, HIPAA-compliant manner on the cloud.

According to some embodiments, caregivers may customize their EMRs to suit their unique needs and evolving medical practices. Optionally, caregivers may access the system through simple conversational commands. Accessing summaries, reports, and forms from the EMR may be through simple conversations with the virtual caregiver avatar. Advantageously, the system may make EMR management accessible, efficient, and patient-centric.

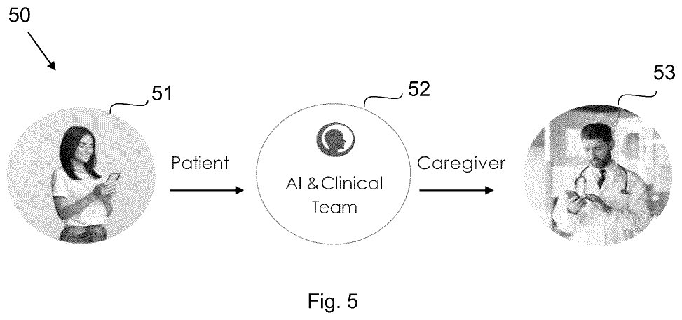

FIG. 5 is a block diagram describing a caregiver avatar system, in accordance with some embodiments of the current invention. For example, the caregiver avatar system may be tailored for the healthcare sector by assisting caregivers.

According to some embodiments, medical professionals may delegate routine actions and/or initial data collection to the system, allowing caregivers to focus more on patient care. The system may include an AI, which may support caregivers and their teams without making decisions for them. The system may handle time-consuming tasks, allowing caregivers to focus on critical care. The system may be fully compliant with medical guidelines, it may use various information sources to provide informed suggestions to the caregiver. The system may enable multitasking with multiple patients, aiming to boost caregiver revenue and improve patient care.

According to some embodiments, the users' facing platform 51 may be supported by a ‘Back Office’ system 52. The back-office system may assist the caregiver 53 in various activities, such as data collection, data analysis, operational activities, etc. For example, a caregiver may be able to view and/or oversee all of their patients, in the case of a clinic, staff members may be able to view and/or oversee all patients within the clinic. The system may be able to add instructions to the AI's behavior in accordance with a specific caregivers' requests. The system may be able to add medical data to update the AI regarding the medical history of the specific patient, and/or maintain the data up to date.

According to some embodiments, users may have the ability to monitor the system's performance, which helps them to maintain control and obtain valuable insights. The system may include controls and/or analytics in general, without sharing sensitive information without patient consent.

For example, in system 50, patient 51 may be evaluated using a virtual avatar. This avatar engages in conversation with the patient about a specific health issue. As patient 51 answers the avatar's questions, caregiver 53 may provide the avatar with additional relevant questions to help narrow down the diagnosis. Caregiver 53 may respond to patient 51 through the avatar. After the conversation, the avatar summarizes the results, which may include possible diagnosis and recommendations for actions to be taken.

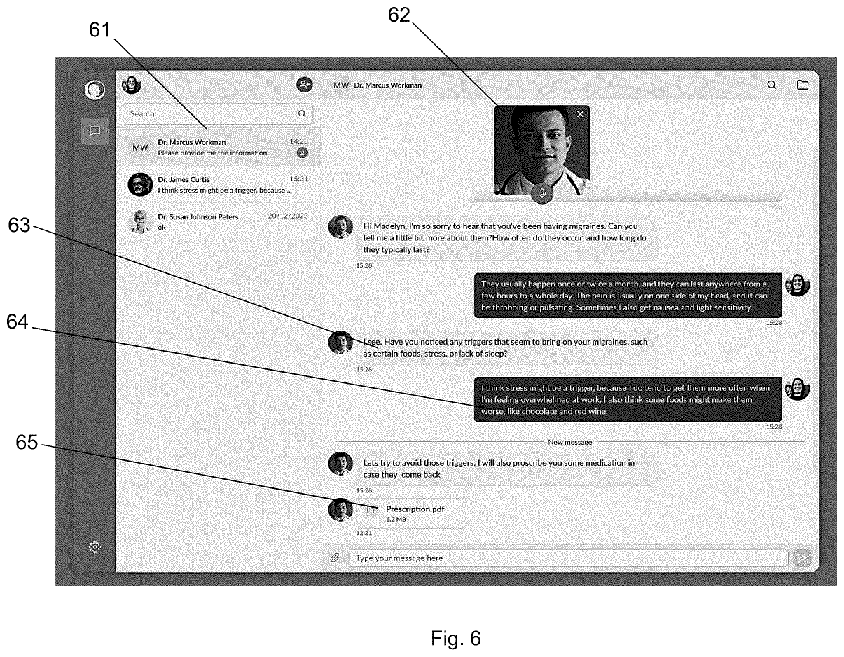

FIG. 6 is a schematic illustration of the system, in accordance with some embodiments. For example, the system may include a list of caregivers 61, from which the patient may select their caregiver. Optionally, the caregivers present in the list accessible to the patient are caregivers on call, caregivers previously consulted by the patient, caregivers with whom a face-to-face appointment has been scheduled (e.g., in the past and/or future). Optionally, a patient may search for a caregiver, e.g., by name, field, specialty, location, etc. Optionally, the caregivers may be ranked by patients. Optionally, the caregivers with the highest rank may appear higher up on the list. Optionally, the ranking may be based on a combined patient satisfaction score. The system may include an image of the avatar and/or caregiver 62. Optionally, the interface may facilitate haptic, audio and/or visual communication between the caregiver (e.g., through the avatar) and the patient. Optionally, a transcript and/or summary of the communication may be presented in the interface. Optionally, caregiver communications 64 may be differentiated from patient communication 63. Optionally, the interface may facilitate transfer of information files 65, such as prescriptions, photographs, test results, videos, remote sensor data, etc.

According to some embodiments, the system may be a cross platform product. Optionally, the system may work on the internet, Android systems, Apple systems, IOS, desktops, laptops, tablets, smart phones, smart devices, virtual reality devices, etc. Optionally, the system may be synchronized in real time across platforms. Optionally, the system interface may be suitable for patient facing and/or caregiver facing interactions.

According to some embodiments, the system may facilitate pre-visit data collection. Talking to a caregiver involves following a standard procedure of sharing personal information about a patient's personal health. The system may be configured to allow patients to share personal information about their health through a video call with an AI Interactive avatar, or by chat.

According to some embodiments, the system may be configured to assist during a caregiver and patient session. Optionally, the appointment with the caregiver may be conducted in person or through telemedicine. Optionally, prior to the appointment session, the system may collect all relevant data from the patient. Optionally, the system may include support for telemedicine.

According to some embodiments, the system may be configured to facilitate follow-ups with the patient. Optionally, the monitoring (follow-up) phase may be vital for chronic conditions, though it applies to other medical issues as well. In this phase, a caregiver may want to know if the patient is compliant with the treatment. Additionally, patients frequently have follow up inquiries and/or need consultations, yet caregivers are not always accessible. The system may provide round-the-clock assistance, catering to patients' needs with the level of care congruent with a caregiver. Optionally, the system may provide support for the entire spectrum of the medical journey.

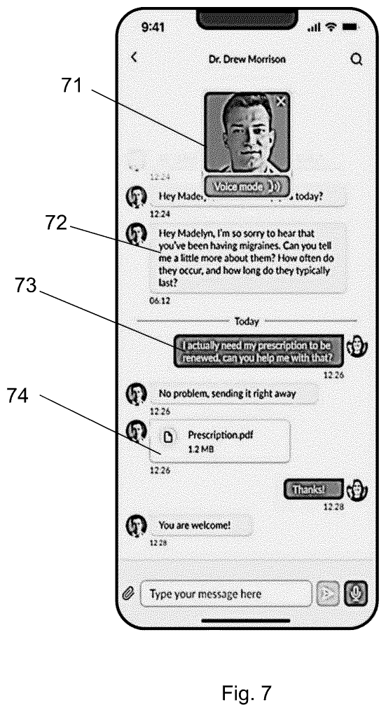

FIG. 7 is a schematic diagram illustrating a patient facing interface, in accordance with some embodiments. For example, the system may include a list of caregivers (not shown), from which the patient may select their caregiver. The system may include an image of the avatar and/or caregiver 71. Optionally, the interface may facilitate haptic, audio and/or visual communication between the caregiver (e.g., through the avatar) and the patient. Optionally, a transcript and/or summary of the communication may be presented in the interface. Optionally, caregiver communications 72 may be differentiated from patient communication 73. Optionally, the interface may facilitate transfer of information files 74, such as prescriptions, photographs, test results, videos, remote sensor data, etc.

According to some embodiments, the patient facing interface may include access to the avatars of all the patient's caregivers, e.g., nurses, doctors, specialists, consultants, etc. Optionally, the patient facing interface may include the history of all previous conversations. Optionally, the patient facing interface may include the summaries of the previous conversations. Optionally, the patient facing interface may include test results, medical history, current medical condition, prescriptions, scheduled appointments and/or test, etc.

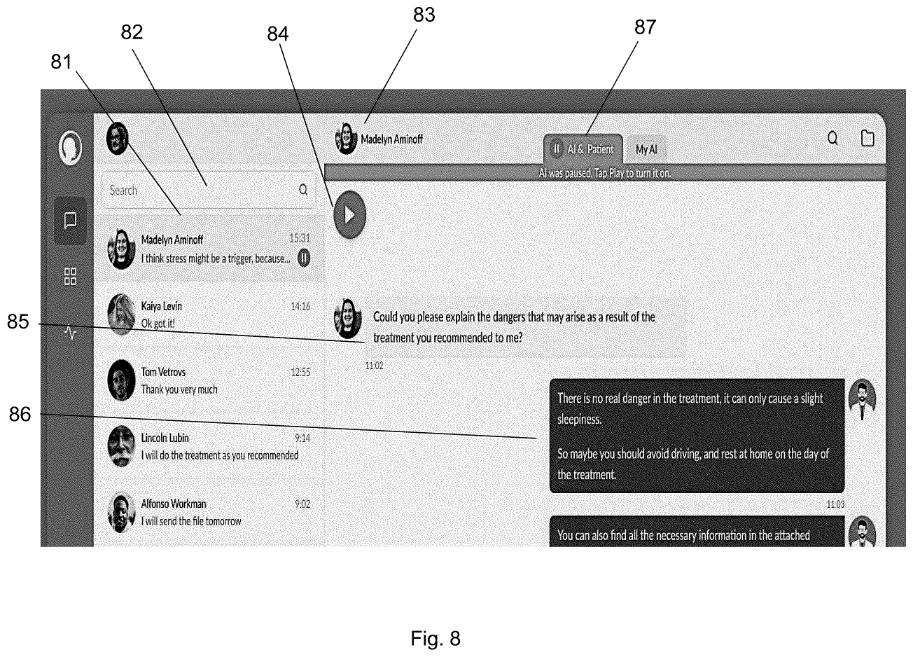

FIG. 8 is a schematic diagram illustrating a caregiver facing interface, in accordance with some embodiments. Optionally, the system may allow multiple views, for example an AI view 87 wherein the doctor may get data and/or give instructions to the AI system (for example general instructions that the system will translate into positive actions, for example “investigate for signs of heart trouble,” and/or receive conversation summary). For example, the system may include a patient interaction view wherein the caregiver can review the queries and response of the patient. For example, the system may include a list of patients 81, from which the caregiver may select a patient to treat and/or a case to review. Optionally, the patients present in the list accessible to the caregiver are patients who previously consulted the caregiver, patients with whom a face-to-face appointment has been scheduled (e.g., in the past and/or future). Optionally, a caregiver may search 82 for a patient, e.g., by name, field, case number, tests performed, etc. Optionally, the system may include an image of the avatar and/or caregiver. Optionally, the system may include an image of patient 83. Optionally, the interface may facilitate haptic, audio and/or visual communication between the caregiver (e.g., through the avatar) and the patient. Optionally, a transcript and/or summary of the communication may be presented in the interface. Optionally, caregiver communications 85 may be differentiated from patient communication 86. Optionally, the interface may facilitate transfer of information files 84, such as prescriptions, photographs, test results, videos, remote sensor data, explanatory information, animations, etc. Optionally, the interface may include one or more tabs from which a caregiver may access various data regarding the patient, e.g., medical history, test results, etc. Optionally, the interface may be configured to facilitate the caregiver changing the avatar interaction mode (e.g., automatic mode, semi-automatic mode, manual mode, etc.). Optionally, the caregiver may interact with the patient through the avatar. Optionally, the caregiver may interact with the patient directly. Optionally, the system may present and/or differentiate between direct communication with the patient, communication with the patient through the Avatar, instructions from the caregiver to the Avatar and/or communication of the caretaker directly to the patient.

According to some embodiments, the caregiver facing interface may include the histories of all previous conversations. Optionally, the caregiver facing interface may include the summaries of the previous conversations. Optionally, the caregiver facing interface may include test results, medical history, current medical condition, prescriptions, scheduled appointments and/or test, etc. Optionally, the caregiver interface may include access to the patient's conversations and/or summaries thereof with other caregivers and/or their avatars. Optionally, the caregiver interface may facilitate synchronous and/or asynchronous monitoring, and/or access, and/or review of avatar-patient interactions. Optionally, the caregiver interface may facilitate addition of questions, information, explanations, and/or amendment of diagnoses. Optionally, the caregiver interface may facilitate real-time interaction with the patient through the avatar.

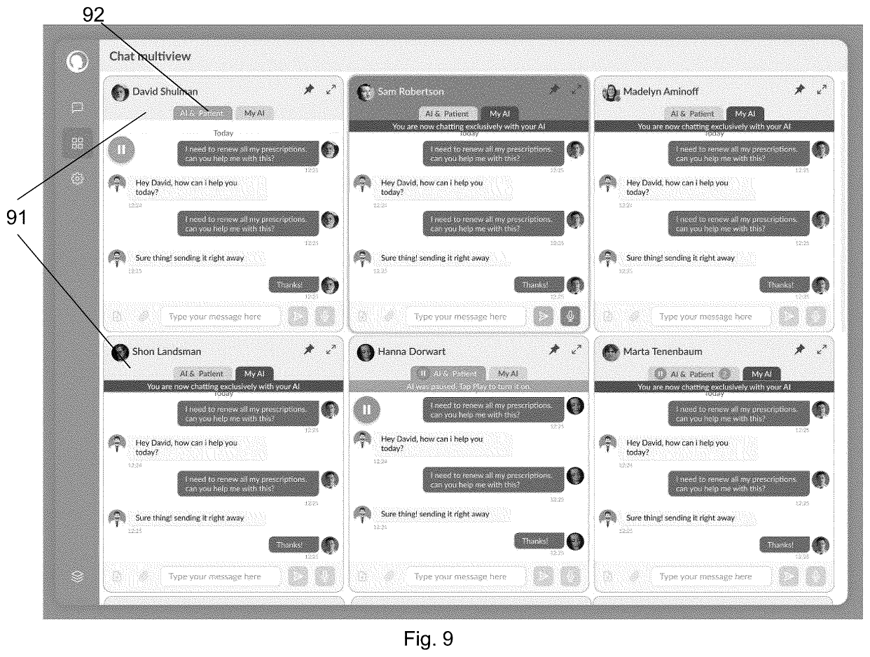

FIG. 9 is a schematic diagram illustrating a caregiver multi-view interface, in accordance with some embodiments. For example, the caregiver multi-view interface may facilitate synchronous and/or asynchronous monitoring, and/or access, and/or review of multiple avatar-patient interactions 91 separately and/or simultaneously. Optionally, the interface may be configured to facilitate the caregiver changing the avatar interaction mode in each of the avatar-patient interactions separately 92 and/or simultaneously (e.g., automatic mode, semi-automatic mode, manual mode, etc.). Optionally, the caregiver interface may facilitate addition of questions, information, explanations, and/or amendment of diagnoses to each of the avatar-patient interactions simultaneously. Optionally, the caregiver interface may facilitate real-time interaction with each patient through the avatar separately and/or simultaneously. Optionally, each one of the multi-view caregiver facing interfaces may include the histories of all previous conversations. Optionally, each one of the multi-view caregiver facing interfaces may include the summaries of the previous conversations. Optionally, each one of the multi-view caregiver facing interfaces may include test results, medical history, current medical condition, prescriptions, scheduled appointments and/or test, etc. Optionally, each one of the multi-view caregiver facing interfaces may include access to the patient's conversations and/or summaries thereof with other caregivers and/or their avatars.

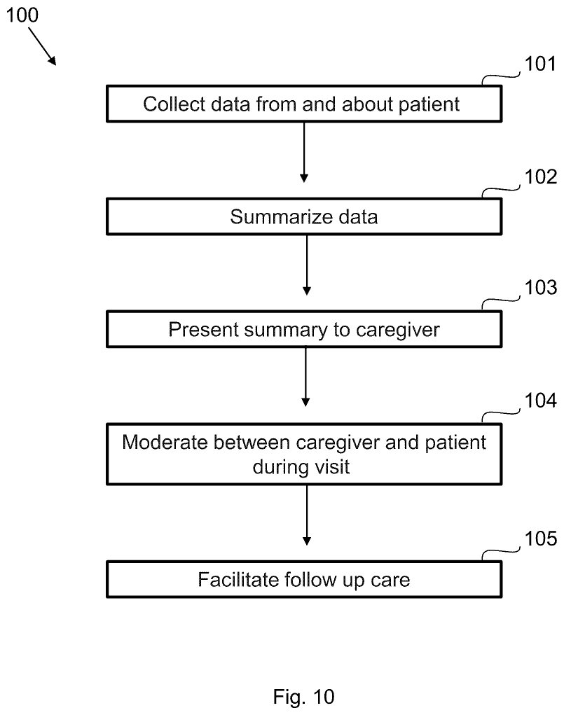

FIG. 10 is a flow diagram of a method of use of a caregiver-patient interface, in accordance with some embodiments. For example, in method 100, an AI system collects data 101 from a and about a patient, e.g., through an interview, electronic medical records, test results, etc. The system summarizes 102 the collected data for a caregiver. The summary may include proposed diagnoses and/or suggestions for future actions. The summary is then presented 103 to the caregiver. The system may moderate 104 between the caregiver and the patient during a visit. Optionally, the visit may be face to face, in person, on-line, etc. The system may facilitate 105 follow up care after the visit, e.g., to facilitate patient compliance, to schedule any required tests, answer any patient queries, etc.

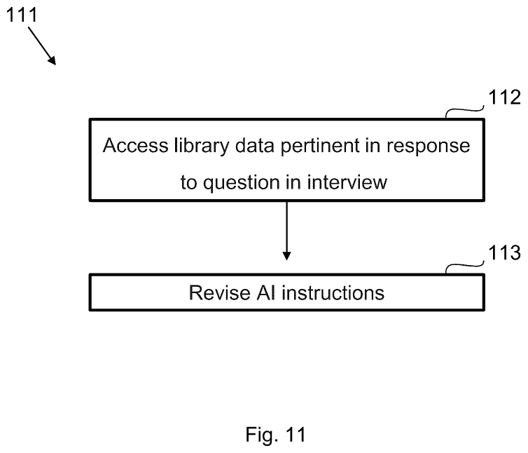

FIG. 11 is a flow diagram of a method of use of a caregiver-patient interface, in accordance with some embodiments. For example, in method 111, the AI system accesses 112 a library specific to a patient condition using a specific prompt file in accordance with predetermined conversation cues received during an avatar-patient interview. AI instructions, training set and/or dataset are revised 113 by giving the AI a specific set of instructions that change according to the cues of the interview and/or instructions of a caregiver and/or other events.

These embodiments are provided by way of example and are in no means intended to limit the scope of the invention.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

### General

It is expected that during the life of a patent maturing from this application many relevant building technologies, artificial intelligence methodologies, computer user interfaces, image capture devices will be developed and the scope of the terms for design elements, analysis routines, user devices is intended to include all such new technologies a priori.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Data and/or program code may be accessed and/or shared over a network, for example the Internet. For example, data may be shared and/or accessed using a social network. A processor may include remote processing capabilities for example available over a network (e.g., the Internet). For example, resources may be accessed via cloud computing. The term “cloud computing” refers to the use of computational resources that are available remotely over a public network, such as the internet, and that may be provided for example at a low cost and/or on an hourly basis. Any virtual or physical computer that is in electronic communication with such a public network could potentially be available as a computational resource. To provide computational resources via the cloud network on a secure basis, computers that access the cloud network may employ standard security encryption protocols such as SSL and PGP, which are well known in the industry.

Some of the methods described herein are generally designed only for use by a computer, and may not be feasible or practical for performing purely manually, by a human expert. A human expert who wanted to manually perform similar tasks might be expected to use completely different methods, e.g., making use of expert knowledge and/or the pattern recognition capabilities of the human brain, which would be vastly more efficient than manually going through the steps of the methods described herein.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise.

As used herein, the term “Photoplethysmography” (PPG) may relate to a simple and low-cost optical technique that can be used to detect blood volume changes in the microvascular bed of tissue. PPG is often used non-invasively to make measurements at the skin surface. The PPG technology has been used in a wide range of commercially available medical devices for measuring oxygen saturation, blood pressure and cardiac output, assessing autonomic function and/or detecting peripheral vascular disease.

As used herein, the term “caregiver” may relate to a physician, nurse, physical therapist, psychologist, behavior therapist, and/or advanced medical care giver.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A system for interactions between a caregiver and a subject comprising:
a remote interface for the subject;
a remote interface for a caregiver;
a processor to control an avatar including
a Large Language Model (LLM) for conversation and
an Artificial Intelligence (AI) module for instructing the LLM and to control a virtual avatar to simulate the caregiver interactions with a subject including collecting clinical data and
evaluating the clinical data for medical conditions.

2-3. (canceled)

4. The system of claim 1, wherein said processor is configured to simulate questions and responses of each particular caregiver of a plurality of caregivers.

5-6. (canceled)

7. The system according to claim 4, wherein the avatar is configured to appear in a video call with appropriate facial expression and voice intonations.

8-10. (canceled)

11. The system according to claim 1, wherein the remote interface for the caregiver includes tools to monitor, assess, and interrupt subject-avatar interactions in real-time.

12-13. (canceled)

14. The system according to claim 11, wherein the AI module is configured to receive simple instructions from the caregiver and in response to said simple instructions trigger complex avatar actions selected from a group consisting of writing prescriptions and sending them to the subject, scheduling tests, scheduling procedures, scheduling a visit with the avatar, scheduling a visit with the caregiver, sending a medication to the subject, performing cognitive behavior therapy sessions with the subject, sending textual, verbal or using visual media instructions and other information to the subject, sending an article of medical equipment to the subject, sending a secondary caretaker to the subject, sending another caregiver to the subject, scheduling physiotherapy, scheduling a medical test, managing a medical test, managing blood sampling and blood testing, organizing transportation to or from a medical facility, scheduling home tests including blood tests, and home treatments.

15. The system according to claim 1, wherein the clinical data includes at least one of, a medical history, an electronic medical record, a hard copy medical record, current health issues, symptoms, test results, remote sensor readings, appearance of skin, body shape, appearance of eyes, appearance of a mouth, facial features, a facial expression, a mood, behaviour, a speech parameter, a breathing parameter, a sound and a gait.

16. (canceled)

17. The system of claim 1, wherein the processor is configured to supervise the subject performing at least one home task selected from sending instructions and getting feedback from the subject, in front of at least one of a camera and a microphone, the at least one home task chosen from: measuring a physiologic parameter using a home device, taking medication, walking, climbing stairs, moving a body part, performing a facial gesture, breathing, and saying a specific sentence.

18-22. (canceled)

23. The system of claim 1, wherein the remote interface for the caregiver is configured to facilitate direct participation of the caregiver in a conversation between the subject and the avatar.

24. The system of claim 1, wherein the remote interface for the caregiver is configured to facilitate silencing the avatar while the caregiver converses with the subject.

25. The system of claim 1, wherein the remote interface for the caregiver is configured to facilitate a caregiver to participate and monitor conversations with a plurality of subjects simultaneously.

26. The system of claim 1, wherein the remote interface for the caregiver is configured to facilitate a plurality of caregivers to converse with the at least one of the avatar and the subject during the same conversation regarding a particular subject.

27. The system of claim 1, wherein the remote interface for the caregiver is configured to facilitate the caregiver conversing with the avatar to add more information regarding the subject from a direct interaction between the subject and the caregiver.

28-29. (canceled)

30. The system of claim 1, wherein the remote interface for the caregiver is configured to facilitate recording a face to face or virtual conversation of the subject with the caregiver and identify who said what and add this data to a summary accordingly.

31-35. (canceled)

36. A method for a caregiver-subject interface, the method comprising:
collecting pre-visit subject data;
summarizing patient data for a caregiver, wherein the summary includes a proposed diagnosis and recommendations for future actions;
assisting in the subject visit; and
ensuring follow-up care of the subject.

37. The method according to claim 36, wherein the collecting subject data comprises actively interviewing the subject, administering tests to the subject, collecting data from subject associated devices, and accessing medical records of the subject.

38-39. (canceled)

40. The method according to claim 36, wherein the follow-up care comprises at least one of monitoring a condition of the subject, monitoring subject compliance, scheduling follow up visits, scheduling consultations, scheduling tests, ordering transportation, renewing prescriptions, answering patient inquiries regarding their diagnosis, prognosis, and treatment.

41. (canceled)

42. A method for dynamic injection instruction to an AI avatar system, the method comprising:
accessing a specific library of instructions from a plurality of libraries in accordance to a specific prompt file of in response to conversation cues; and
giving a new set of instructions to the AI according to at least one of conversation cues during an interaction with at least one of a subject and a service provider.

43. The method of claim 42, wherein the conversation is between a subject and an avatar and the conversation cues are from the subject, and the service provider is a caretaker.

44-46. (canceled)

47. The method of claim 42, wherein a specific library of the plurality of libraries includes specific conversation cues from an existing validated database of symptoms and responses for a caregiver interview with a subject having a condition relating to said conversation cues.

48. The method of claim 47, wherein the specific library is customized for a specific caregiver and includes cues from recorded conversations of the specific caregiver.

49-50. (canceled)
